(19) United States
(12) Patent Application Publication
(10) Pub. No.: US 2010/0036136 A1
(43) Pub. Date: Feb. 11, 2010

(54) Maleimide Containing Ester and Sulfide Functionalities

(76) Inventor: Osama M. Musa, Hillsborough, NJ (US)

Correspondence Address: Henkel Corporation, 10 Finderne Avenue, Bridgewater, NJ, 08807, US

(21) Appl. No.: 12/537,541

(22) Filed: Aug. 7, 2009

Related U.S. Application Data
(63) Continuation of application No. PCT/US2007/003422, filed on Feb. 8, 2007.

(51) Int. Cl. C07D 401/14; C07F 7/02

(52) U.S. Cl. 548/521; 548/406

(57) ABSTRACT

An oligomeric compound that is the Michael addition reaction product of a thiol with a compound having maleimide terminal groups and ester functionality internal in the compound can be formulated into curable compositions to improve adhesion to metal substrates.

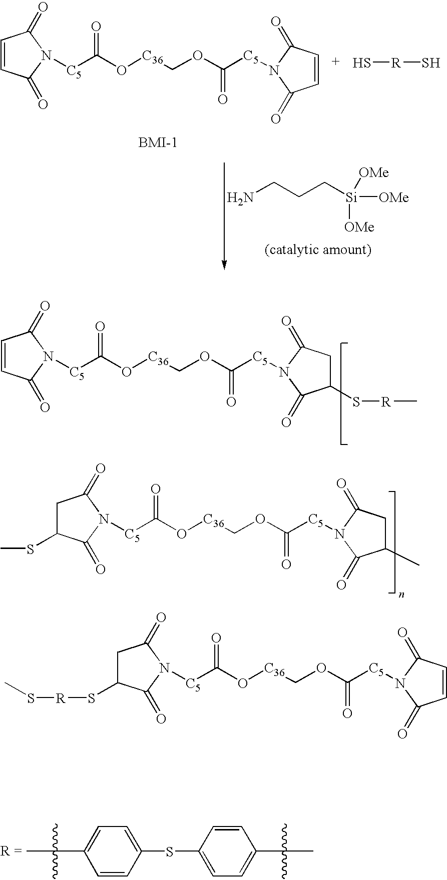

### CROSS-REFERENCE TO RELATED APPLICATION

This application is a continuation of International Application No. PCT/US2007/003422 filed Feb. 8, 2007, the contents of which are incorporated herein by reference.

### FIELD OF THE INVENTION

This invention relates to oligomeric compounds containing maleimide, ester, and sulfide functionalities, which compounds are useful as adhesives, coatings, and encapsulants. These compounds are particularly useful for various fabrication steps in semiconductor packaging.

### BACKGROUND OF THE INVENTION

Adhesives for use on metal, glass, and plastic surfaces have many applications within various industries. Adhesion to these surfaces in general is difficult and new compounds or formulations are sought for both quick and strong adherence. Such materials would be particularly useful within the semiconductor packaging industry. Common steps in the fabrication of semiconductor packages involve affixing semiconductor devices onto substrates or encapsulating or coating parts or all of the device. The more prominent steps that use adhesives, coatings or encapsulants are the bonding of integrated circuit chips to lead frames or other substrates, the bonding of circuit packages or assemblies to printed wire boards, the encapsulation of solder balls used as electrical connections, and the coating of via holes. In these applications, the components of the package are prepared from different materials, such as metal, glass, silicon, and plastic, and the adhesive or encapsulant must bond to the surface of each. Moreover, the adhesive or encapsulant must maintain its bond to both materials through temperature and humidity cycles. Thus, there is always a need for new compounds and formulations within the semiconductor packaging industry and within other industries using components that must adhere to more than one type of surface.

### SUMMARY OF THE INVENTION

This invention is directed to oligomeric compounds that contain (i) maleimide functionality, (ii) sulfide functionality, and (iv) ester functionality. In a further embodiment, this invention is directed to curable compositions, such as, adhesives, coatings, and encapsulants, that contain such oligomeric compounds.

### DETAILED DESCRIPTION OF THE INVENTION

As used within this specification and the claims, a thiol, also known as a mercaptan, has the structure R—SH and a sulfide has the structure R—S—R in which R is an aliphatic or aromatic moiety, with or without heteroatoms.

The oligomers of this invention are synthesized from the Michael addition reaction of the double bond in a maleimide moiety with the —SH in a thiol. The maleimide starting compound has the structure:

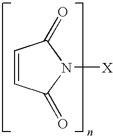

in which n is an integer, preferably an integer from 1 to 3, and X is an aliphatic or aromatic moiety that contains at least one ester functionality.

Exemplary X moieties include

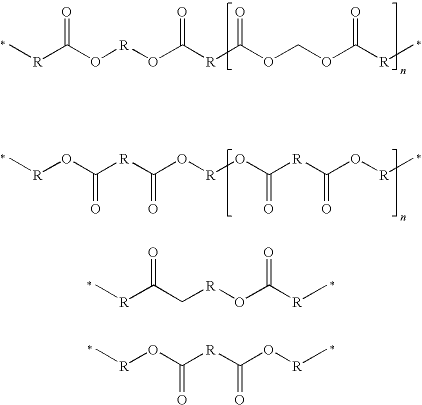

in which each R is independently an aliphatic or aromatic moiety, with or without heteroatoms, or a siloxane having the structure

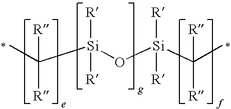

in which R″ is independently for each position a H or an alkyl group having 1 to 5 carbon atoms, and R^{′} is independently for each position an alkyl group having 1 to 5 carbon atoms or an aryl group, and e and f independently are an integer from 1 to 10 and g is an integer from 1 to 50.

The asterisks in the above structures indicate the bond connecting the X moiety to the maleimide functionality in the maleimide starting material.

The thiol compounds used to react with the maleimide are linear or branched mercaptans with two or more thio-groups. Exemplary mercaptans (thiols) include, but are not limited to: 2-mercapto ethyl sulfide [(HSCH₂CH₂)₂S]; triethylene glycol dimercaptan and similar polyglycol dimercaptans and similar di-olefin dimercaptans; ethylcyclohexyl dimercaptan; ortho-, meta-, and para-benzene dithiol; ortho-, meta-, and para-benzene dimethane thiol [o-, m- and p-(HSCH₂)₂C₆H₄]; pentaerythrityl tetrathiol; 4,4′-thiobisbenzene thiol; thiol terminated polyethers; thiol terminated polythioethers; thiol terminated polyurethanes; and similar multifunctional thiols known in the art.

Other commercially available mercaptans are Capcure 3-800 or Capcure LOF from Cognis Corp.; THIOKOL LP-3 from Rohm and Haas Company, Philadelphia, Pa.; THIOPLAST from Akcros Chemicals, Manchester, Great Britain, including products referred to as G1 (n is 19 21, 1.8 2 percent thiol content, and a 3,300 3,700 molecular weight), G4 (n is less than 7, less than 5.9 percent thiol content, and less than 1,100 molecular weight), G12 (n is 23 26, 1.51.7 percent thiol content, and a 3,900 4,400 molecular weight), G21 (n is 12 15, 2.5 3.1 percent thiol content, and a 2,100 2,600 molecular weight), G22 (n is 14 18, 2.12.7 percent thiol content, and a 2,400 3,100 molecular weight), G112 (n is 23 25, 1.51.7 percent thiol content, and a 3,900 4,300 molecular weight), and G131 (n is 30 38, 1.51.7 percent thiol content, and a 5,000 6,500 molecular weight).

In one embodiment the inventive oligomer (reaction product of the Michael addition of a maleimide and thiol) will have a structure selected from the group consisting of

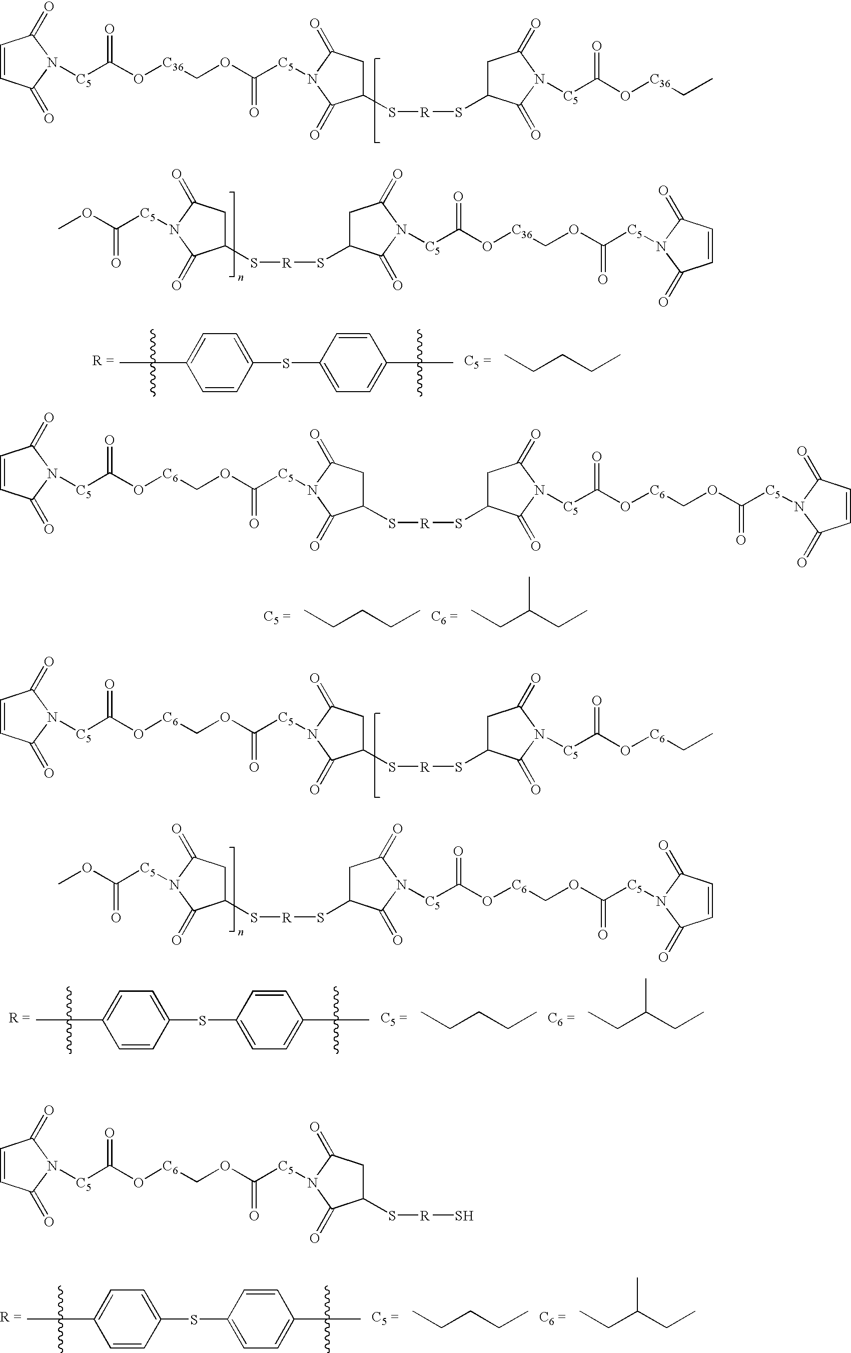

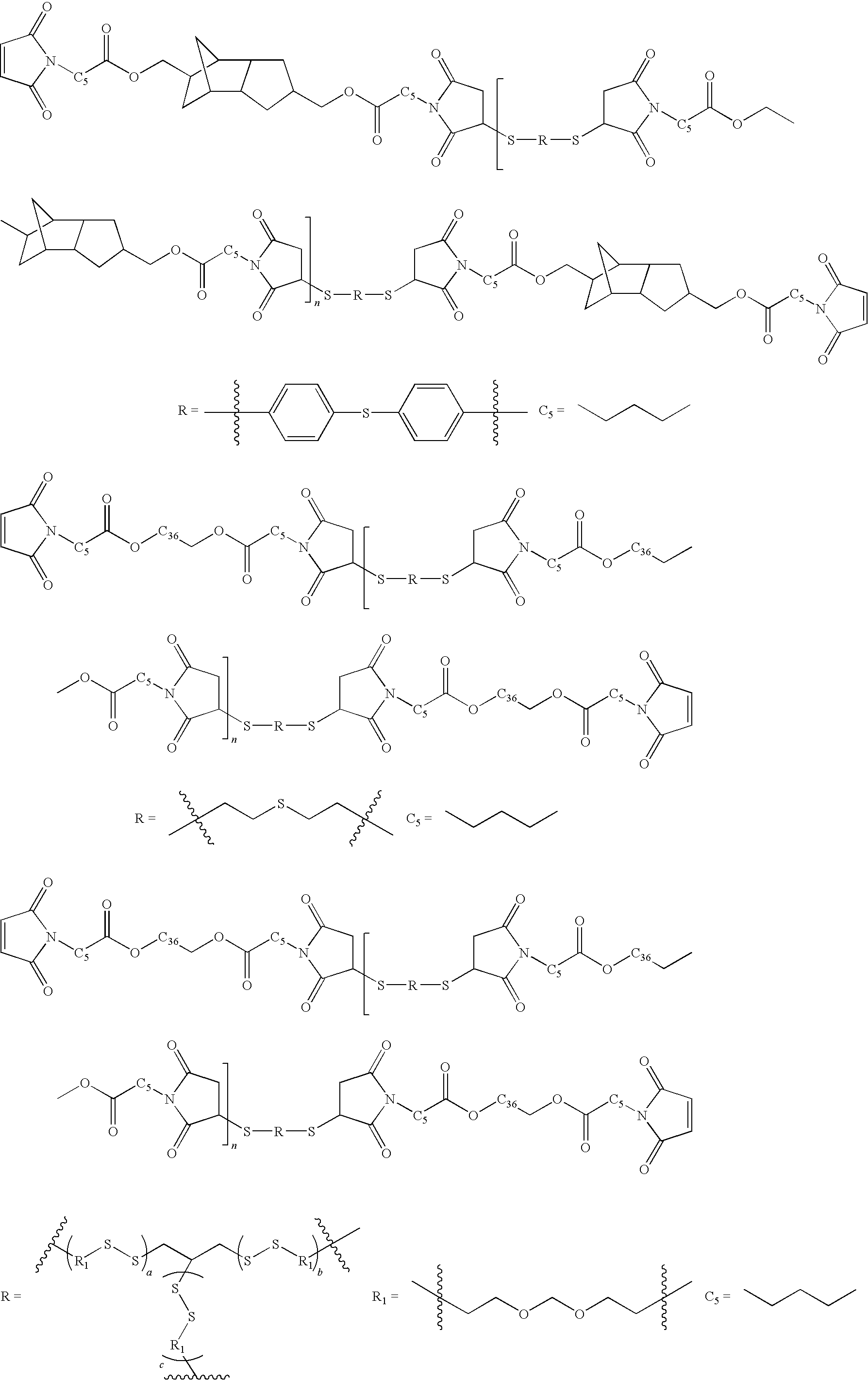

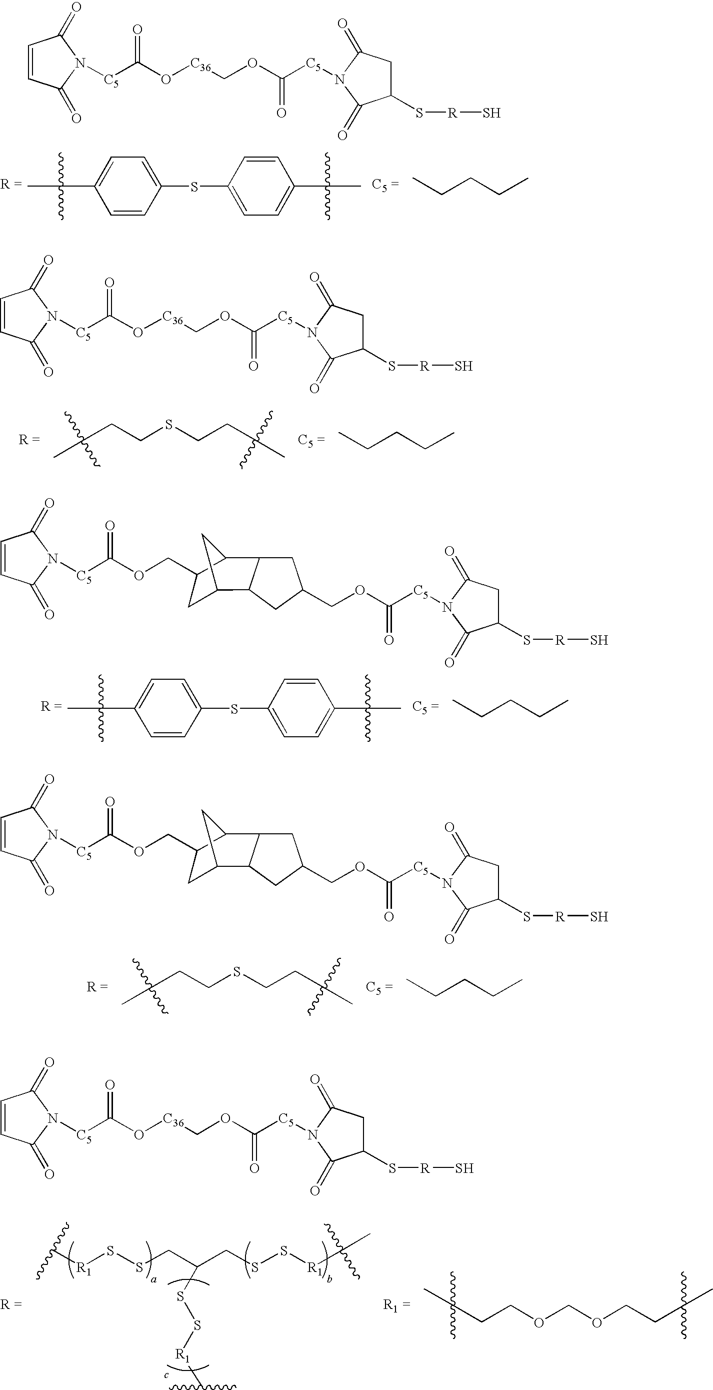

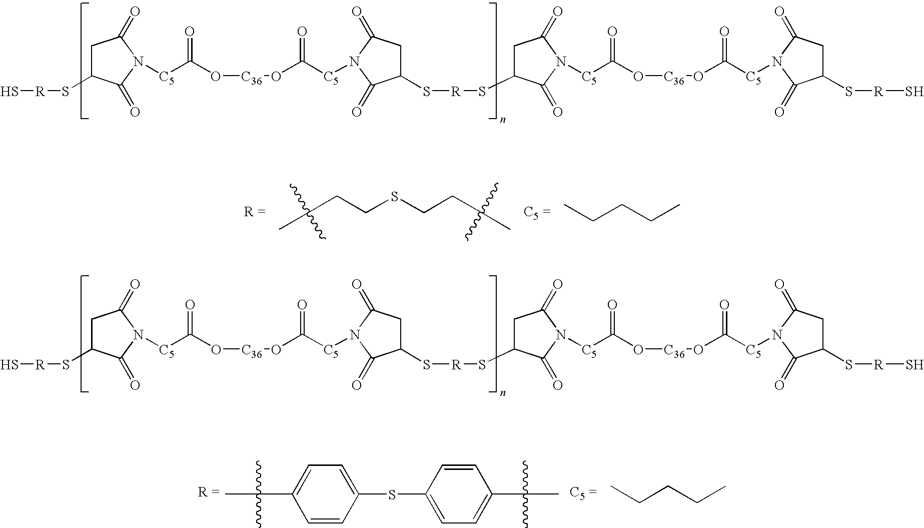

Typical reaction conditions are disclosed in the Examples section of this specification. The stoichiometry of the reaction can range from 1:50 to 50:1 maleimide to thiol. Viscosity increases as the ratio approaches 1:1.

The reaction product of those reactions in which there is an excess of maleimide starting compound will be mainly an oligomer with terminal maleimide groups. The reaction product of those reactions in which there is an excess of thiol will be mainly an oligomer with terminal thiol groups.

For the fabrication of semiconductor packages, preferred adhesives are those with low glass transition temperatures (Tg). Low Tg materials typically do not hold up well under conditions of 85° C. and 85% RH, and consequently adhesion of these materials is poor. To offset this deficiency, adhesives are formulated with adhesion promoters. Sulfur-containing compounds are known to be good adhesion promoters and are efficient at increasing adhesion to metal through the chemical bonding of the S—H group to the metal to form a S-M bond, or through van der Waals interactions between the sulfur atom and the metal.

The S—H group, however, can react with peroxide, a common catalyst for free radical polymerizing formulations, to initiate radical polymerization. Thus, the presence of the S-H group in adhesion formulations may be undesirable for some applications because it could initiate a premature polymerization of the adhesive leading to gelation and short work life. For those applications it is preferable to use an excess of maleimide so that the sulfur linkages are internal within the oligomer and not terminal S—H groups, which would be the case if an excess of thiol is used in the synthesis of the oligomer.

The oligomers prepared with an excess of maleimide generally present relatively high viscosity, and may require diluents reactive with the maleimide for ease of application.

Exemplary reactive diluents are liquids with low viscosity and high reactivity with the maleimide functionality in the inventive oligomer, and include, but are not limited to, vinyl acetate, other maleimides, compounds containing a styrene moiety, vinyl ether, maleates and fumarates, and compounds containing a cinnamyl moiety. Examples of such reactive diluents are:

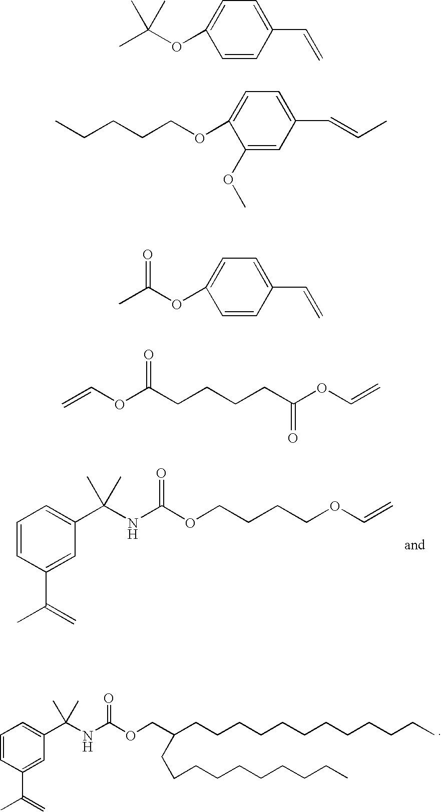

The inventive oligomers may be used as the major component in formulations for adhesives, coatings, or encapsulants, or as a minor additive to such formulations containing other resins as the major component. Whether it is a major or minor component, selection of the remaining materials will be dependent upon the end use application. If other resins are present, typically such resins will be thermoplastics, thermosets, elastomers, thermoset rubbers, or a combination of these. The formulation may or may not contain solvent, and in many cases will contain a filler, which can be present in an amount up to 95% of the total composition.

Other components may be added at the option of the practitioner; such other components include, but are not limited to, curing agents, fluxing agents, wetting agents, flow control agents, adhesion promoters, and air release agents. A curing agent is any material or combination of materials that initiate, propagate, or accelerate cure of the coating and includes accelerators, catalysts, initiators, and hardeners.

The viscosity and thixotropic index of the final formulation will be selected by the practitioner to be suitable for the application method, manufacturing conditions, and end uses, and such selection is within the expertise of one skilled in the art. For instance, if the end use composition is to be applied via spin coating the viscosity of the composition should be fairly low. If the composition will be applied with screen printing, it will generally have a higher viscosity.

Any resins and polymers used in the formulation, in addition to the inventive oligomers, may be solid, liquid, or a combination of the two. Suitable additional resins and polymers include epoxies, acrylates and meth-acrylates, maleimides, bismaleimides, vinyl ethers, polyesters, poly(butadienes), siliconized olefins, silicone resins, siloxanes, styrene resins and cyanate ester resins.

Exemplary solid aromatic bismaleimide (BMI) resin powders for use in formulations with the inventive oligomers, are those having the structure

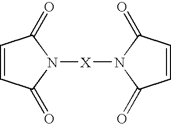

in which X is an aromatic group. Exemplary aromatic groups include:

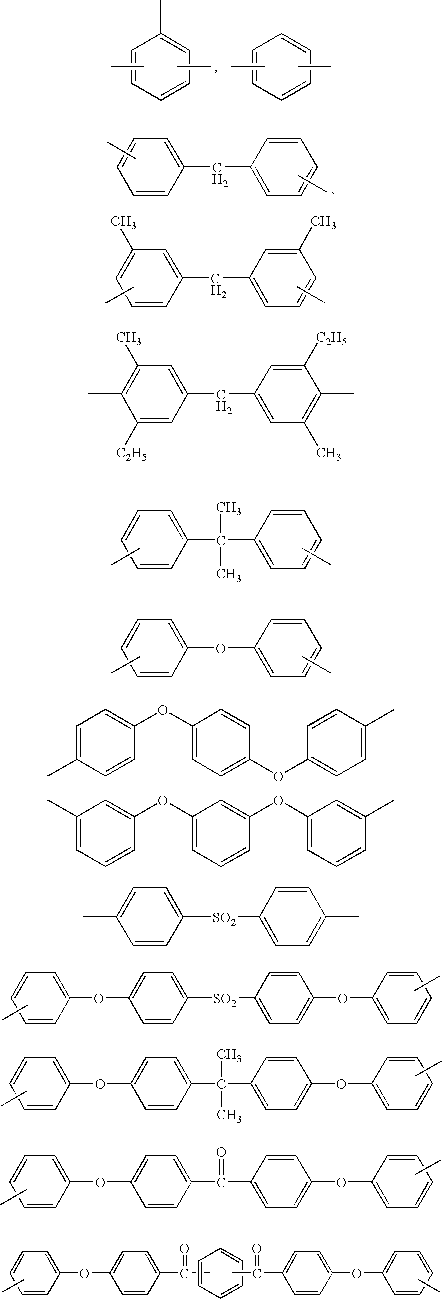

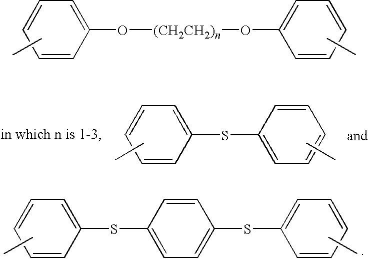

Bismaleimide resins having these X bridging groups are commercially available, and can be obtained, for example, from Sartomer (USA) or HOS-Technic GmbH (Austria).

Additional exemplary maleimide resins for use in formulations with the inventive oligomers include those having the generic structure

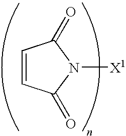

n which n is 1 to 3 and X¹ is an aliphatic or aromatic group. Exemplary X¹ entities include, poly(butadienes), poly(carbonates), poly(urethanes), poly(ethers), poly(esters), simple hydrocarbons, and simple hydrocarbons containing functionalities such as carbonyl, carboxyl, amide, carbamate, urea, or ether. These types of resins are commercially available and can be obtained, for example, from National Starch and Chemical Company and Dainippon Ink and Chemical, Inc.

Specific preferred maleimide resins include

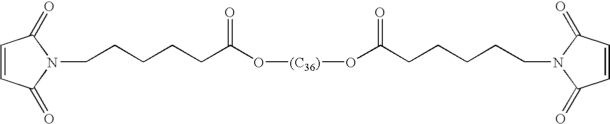

in which C₃₆ represents a linear or branched chain (with or without cyclic moieties) of 36 carbon atoms;

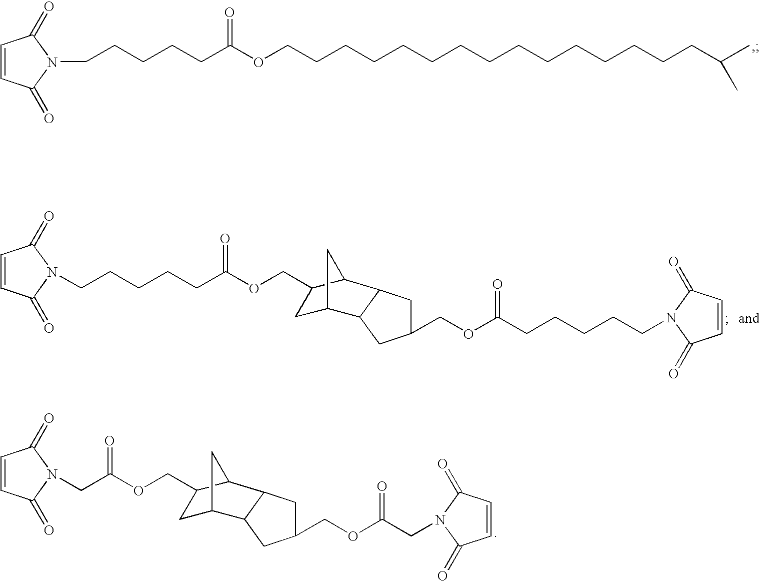

Suitable acrylate resins for use in formulation with the inventive oligomers include those having the generic structure

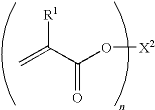

in which n is 1 to 6, R¹ is —H or —CH₃. and X² is an aromatic or aliphatic group. Exemplary X² entities include poly(butadienes), poly(carbonates), poly(urethanes), poly(ethers), poly(esters), simple hydrocarbons, and simple hydrocarbons containing functionalities such as carbonyl, carboxyl, amide, carbamate, urea, or ether. Commercially available materials include butyl (meth)acrylate, isobutyl (meth)acrylate, 2-ethyl hexyl (meth)acrylate, isodecyl (meth)acrylate, n-lauryl (meth)acrylate, alkyl (meth)acrylate, tridecyl (meth)acrylate, n-stearyl (meth)acrylate, cyclohexyl(meth)acrylate, tetrahydrofurfuryl(meth)acrylate, 2-phenoxy ethyl(meth)acrylate, isobornyl(meth)acrylate, 1,4-butanediol di(meth)acrylate, 1.6 hexanediol di(meth)acrylate, 1,9-nonandiol di(meth)acrylate, perfluorooctylethyl (meth)acrylate, 1,10 decandiol di(meth)acrylate, nonylphenol polypropoxylate (meth)acrylate, and polypentoxylate tetrahydrofurfuryl acrylate, available from Kyoeisha Chemical Co., LTD; polybutadiene urethane dimethacrylate (CN302, NTX6513) and polybutadiene dimethacrylate (CN301, NTX6039, PRO6270) available from Sartomer Company, Inc; polycarbonate urethane diacrylate (ArtResin UN9200A) available from Negami Chemical Industries Co., LTD; acrylated aliphatic urethane oligomers (Ebecryl 230, 264, 265, 270,284, 4830, 4833, 4834, 4835, 4866, 4881, 4883, 8402, 8800-20R, 8803, 8804) available from Radcure Specialities, Inc; polyester acrylate oligomers (Ebecryl 657, 770, 810, 830, 1657, 1810, 1830) available from Radcure Specialities, Inc.; and epoxy acrylate resins (CN104, 111, 112, 115, 116, 117, 118, 119, 120, 124, 136) available from Sartomer Company, Inc. In one embodiment the acrylate resins are selected from the group consisting of isobornyl acrylate, isobornyl methacrylate, lauryl acrylate, lauryl methacrylate, poly(butadiene) with acrylate functionality and poly(butadiene) with methacrylate functionality.

Suitable vinyl ether resins for use in formulations with the inventive oligomers include those having the generic structure

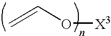

in which n is 1 to 6 and X³ is an aromatic or aliphatic group. Exemplary X³ entities include poly(butadienes), poly(carbonates), poly(urethanes), poly(ethers), poly(esters), simple hydrocarbons, and simple hydrocarbons containing functionalities such as carbonyl, carboxyl, amide, carbamate, urea, or ether. Commercially available resins include cyclohenanedimethanol divinylether, dodecylvinylether, cyclohexyl vinylether, 2-ethylhexyl vinylether, dipropyleneglycol divinylether, hexanediol divinylether, octadecylvinylether, and butandiol divinylether available from International Speciality Products (ISP); Vectomer4010, 4020, 4030, 4040, 4051, 4210, 4220, 4230, 4060, 5015 available from Sigma-Aldrich, Inc.

Suitable poly(butadiene) resins for use in formulations with the inventive oligomers include poly(butadienes), epoxidized poly(butadienes), maleic poly(butadienes), acrylated poly(butadienes), butadiene-styrene copolymers, and butadiene-acrylonitrile copolymers. Commercially available materials include homopolymer butadiene (Ricon130, 131, 134, 142, 150, 152, 153, 154, 156, 157, P30D) available from Sartomer Company, Inc; random copolymer of butadiene and styrene (Ricon 100, 181, 184) available from Sartomer Company Inc.; maleinized poly(butadiene) (Ricon 130MA8,130MA13, 130MA20, 131MA5, 131MA10, 131MA17, 131MA20, 156MA17) available from Sartomer Company, Inc.; acrylated poly(butadienes) (CN302, NTX6513, CN301, NTX6039, PRO6270, Ricacryl 3100, Ricacryl 3500) available from Sartomer Inc.; epoxydized poly(butadienes) (Polybd 600, 605) available from Sartomer Company. Inc. and Epolead PB3600 available from Daicel Chemical Industries, Ltd; and acrylonitrile and butadiene copolymers (Hycar CTBN series, ATBN series, VTBN series and ETBN series) available from Hanse Chemical.

Suitable epoxy resins for use in formulations containing the inventive oligomers include bisphenol, naphthalene, and aliphatic type epoxies. Commercially available materials include bisphenol type epoxy resins (Epiclon 830LVP, 830CRP, 835LV, 850CRP) available from Dainippon Ink & Chemicals, Inc.; naphthalene type epoxy (Epiclon HP4032) available from Dainippon Ink & Chemicals, Inc.; aliphatic epoxy resins (Araldite CY179, 184, 192, 175, 179) available from Ciba Specialty Chemicals, (Epoxy 1234, 249, 206) available from Union Carbide Corporation, and (EHPE-3150) available from Daicel Chemical Industries, Ltd. Other suitable epoxy resins include cycloaliphatic epoxy resins, bisphenol-A type epoxy resins, bisphenol-F type epoxy resins, epoxy novolac resins, biphenyl type epoxy resins, naphthalene type epoxy resins, dicyclopentadiene-phenol type epoxy resins, reactive epoxy diluents, and mixtures thereof.

Suitable siliconized olefin resins for use in the formulations containing the inventive oligomers are obtained by the selective hydrosilation reaction of silicone and divinyl materials, having the generic structure,

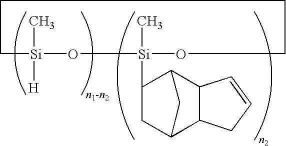

in which n₁ is 2 or more, n₂ is 1 or more and n₁>n₂. These materials are commercially available and can be obtained, for example, from National Starch and Chemical Company.

Suitable silicone resins for use in formulations with the inventive oligomers include reactive silicone resins having the generic structure

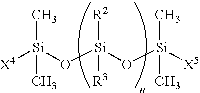

in which n is 0 or any integer, X⁴ and X⁵ are hydrogen, methyl, amine, epoxy, carboxyl, hydroxy, acrylate, methacrylate, mercapto, phenol, or vinyl functional groups, R² and R³ can be —H, —CH₃, vinyl, phenyl, or any hydrocarbon structure with more than two carbons. Commercially available materials include KF8012, KF8002, KF8003, KF-1001, X-22-3710, KF6001, X-22-164C, KF2001, X-22-170DX, X-22-173DX, X-22-174DX X-22-176DX, KF-857, KF862, KF8001, X-22-3367, and X-22-3939A available from Shin-Etsu Silicone International Trading (Shanghai) Co., Ltd.

Suitable styrene resins for use in formulations with the inventive oligomers include those resins having the generic structure

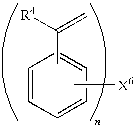

in which n is 1 or greater, R⁴ is —H or —CH₃, and X⁶ is an aliphatic group. Exemplary X³ entities include poly(butadienes), poly(carbonates), poly(urethanes), poly(ethers), poly(esters), simple hydrocarbons, and simple hydrocarbons containing functionalities such as carbonyl, carboxyl, amide, carbamate, urea, or ether. These resins are commercially available and can be obtained, for example, from National Starch and Chemical Company or Sigma-Aldrich Co.

Suitable cyanate ester resins for use in formulations with the inventive oligomers include those having the generic structure

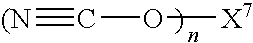

in which n is 1 or larger, and X⁷ is a hydrocarbon group. Exemplary X⁷ entities include bisphenol, phenol or cresol novolac, dicyclopentadiene, polybutadiene, polycarbonate, polyurethane, polyether, or polyester. Commercially available materials include; AroCy L-10, AroCy XU366, AroCy XU371, AroCy XU378, XU71787.02L, and XU 71787.07L, available from Huntsman LLC; Primaset PT30, Primaset PT30 S75, Primaset PT60, Primaset PT60S, Primaset BADCY, Primaset DA230S, Primaset MethylCy, and Primaset LECY, available from Lonza Group Limited; 2-allyphenol cyanate ester, 4-methoxyphenol cyanate ester, 2,2-bis(4-cyanatophenol)-1,1,1,3,3,3-hexafluoropropane, bisphenol A cyanate ester, diallylbisphenol A cyanate ester, 4-phenylphenol cyanate ester, 1,1,1-tris(4-cyanatophenyl)ethane, 4-cumylphenol cyanate ester, 1,1-bis(4-cyanateophenyl)ethane, 2,2,3,4,4,5,5,6,6,7,7-dodecafluorooctanediol dicyanate ester, and 4,4′-bisphenol cyanate ester, available from Oakwood Products, Inc.

Additional polymers suitable for use in formulations with the inventive oligomers include polyamide, phenoxy, polybenzoxazine, polyether sulfone, polyimide, benzoxazine, vinyl ether, polyolefin, polybenzoxyzole, polyester, polystyrene, polycarbonate, polypropylene, poly(vinyl chloride), polyisobutylene, polyacrylonitrile, poly(methyl methacrylate), poly(vinyl acetate), poly(2-vinylpridine), cis-1,4-polyisoprene, 3,4-polychloroprene, vinyl copolymer, poly(ethylene oxide), poly(ethylene glycol), polyformaldehyde, polyacetaldehyde, poly(b-propiolacetone), poly(10-decanoate), poly(ethylene terephthalate), polycaprolactam, poly(11-undecanoamide), poly(m-phenylene-terephthalamide), poly(tetramethlyene-m-benzenesulfonamide), polyester polyarylate, poly(phenylene oxide), poly(phenylene sulfide), polysulfone, polyimide, polyetheretherketone, polyetherimide, fluorinated polyimide, polyimide siloxane, poly-iosindolo-quinazolinedione, polythioetherimide poly-phenyl-quinoxaline, polyquuinixalone, imide-aryl ether phenylquinoxaline copolymer, polyquinoxaline, polybenzimidazole, polybenzoxazole, polynorbornene, poly(arylene ethers), polysilane, parylene, benzocyclobutenes, hydroxy(benzoxazole) copolymer, poly(silarylene siloxanes), and polybenzimidazole.

Other suitable materials for inclusion in adhesive, coating, and encapsulant compositions containing the inventive oligomers include rubber polymers such as block copolymers of monovinyl aromatic hydrocarbons and conjugated diene, e.g., styrene-butadiene, styrene-butadiene-styrene (SBS), styrene-isoprene-styrene (SIS), styrene-ethylene-butylene-styrene (SEBS), and styrene-ethylene-propylene-styrene (SEPS).

Other suitable materials for inclusion in compositions containing the inventive oligomers include ethylene-vinyl acetate polymers, other ethylene esters and copolymers, e.g., ethylene methacrylate, ethylene n-butyl acrylate and ethylene acrylic acid; polyolefins such as polyethylene and polypropylene; polyvinyl acetate and random copolymers thereof; polyacrylates; polyamides; polyesters; and polyvinyl alcohols and copolymers thereof.

Suitable thermoplastic rubbers for use in formulations containing the inventive oligomers include carboxy terminated butadiene-nitrile (CTBN)/epoxy adduct, acrylate rubber, vinyl-terminated butadiene rubber, and nitrile butadiene rubber (NBR). In one embodiment the CTBN epoxy adduct consists of about 20-80 wt % CTBN and about 20-80 wt % diglycidyl ether bisphenol A: bisphenol A epoxy (DGEBA). A variety of CTBN materials are available from Noveon Inc., and a variety of bisphenol A epoxy materials are available from Dainippon Ink and Chemicals, Inc., and Shell Chemicals. NBR rubbers are commercially available from Zeon Corporation.

Suitable siloxanes for use in formulations containing the inventive oligomers include elastomeric polymers comprising a backbone and pendant from the backbone at least one siloxane moiety that imparts permeability, and at least one reactive moiety capable of reacting to form a new covalent bond. Examples of suitable siloxanes include elastomeric polymers prepared from: 3-(tris(trimethyl-silyloxy)silyl)-propyl methacrylate, n-butyl acrylate, glycidyl methacrylate, acrylonitrile, and cyanoethyl acrylate; 3-(tris(trimethylsilyloxy)silyl)-propyl methacrylate, n-butyl acrylate, glycidyl methacrylate, and acrylonitrile; and 3-(tris(trimethylsilyloxy)silyl)-propyl methacrylate, n-butyl acrylate, glycidyl methacrylate, and cyanoethyl acrylate.

If a curing agent is required, its selection is dependent on the polymer chemistry used and the processing conditions employed. As curing agents, the compositions may use aromatic amines, alycyclic amines, aliphatic amines, tertiary phosphines, triazines, metal salts, aromatic hydroxyl compounds, or a combination of these. Appropriateness of the type and amount of catalyst used for specific compositions is disclosed in the open literature and is within the expertise of one skilled in the art.

Examples of such catalysts include imidazoles, such as 2-methylimidazole, 2-undecylimidazole, 2-heptadecyl imidazole, 2-phenylimidazole, 2-ethyl 4-methylimidazole, 1-benzyl-2-methylimidazole, 1-propyl-2-methylimidazole, 1-cyanoethyl-2-methylimidazole, 1-cyanoethyl-2-ethyl-4-methylimidazole, 1-cyanoethyl-2-undecylimidazole, 1-cyanoethyl-2-phenylimidazole, 1-guanaminoethyl-2-methylimidazole and addition product of an imidazole and trimellitic acid; tertiary amines, such as N,N-dimethyl benzylamine, N,N-dimethylaniline, N,N-dimethyltoluidine, N,N-dimethyl-p-anisidine, p-halogeno-N,N-dimethylaniline, 2-N-ethylanilino ethanol, tri-n-butylamine, pyridine, quinoline, N-methylmorpholine, triethanolamine, triethylenediamine, N,N,N′,N′-tetramethylbutanediamine, N-methylpiperidine; phenols, such as phenol, cresol, xylenol, resorcine, and phloroglucin; organic metal salts, such as lead naphthenate, lead stearate, zinc naphthenate, zinc octolate, tin oleate, dibutyl tin maleate, manganese naphthenate, cobalt naphthenate, and acetyl aceton iron; and inorganic metal salts, such as stannic chloride, zinc chloride and aluminum chloride; peroxides, such as benzoyl peroxide, lauroyl peroxide, octanoyl peroxide, acetyl peroxide, para-chlorobenzoyl peroxide and di-t-butyl diperphthalate; acid anhydrides, such as carboxylic acid anhydride, maleic anhydride, phthalic anhydride, lauric anhydride, pyromellitic anhydride, trimellitic anhydride, hexahydrophthalic anhydride; hexahydropyromellitic anhydride and hexahydrotrimellitic anhydride, azo compounds, such as azoisobutylonitrile, 2,2′-azobispropane, m,m′-azoxystyrene, hydrozones, and mixtures thereof.

Suitable curing accelerators may be selected from the group consisting of triphenylphosphine, alkyl-substituted imidazoles, imidazolium salts, onium salts, quartenary phosphonium compounds, onium borates, metal chelates, 1,8-diazacyclo[5.4.0]undex-7-ene or a mixture thereof.

The curing agent can be either a free radical initiator or cationic initiator, depending on whether a radical or ionic curing resin is chosen. If a free radical initiator is used, it will be present in an effective amount. An effective amount typically is 0.1 to 10 percent by weight of the organic compounds (excluding any filler). Appropriate free-radical initiators include peroxides, such as butyl peroctoates and dicumyl peroxide, and azo compounds, such as 2,2′-azobis(2-methyl-propanenitrile) and 2,2′-azobis(2-methyl-butanenitrile). Preferred cationic curing agents include dicyandiamide, phenol novolak, adipic dihydrazide, diallyl melamine, diamino malconitrile, BF3-amine complexes, amine salts and modified imidazole compounds.

Metal compounds also can be employed as cure accelerators for cyanate ester systems and include, but are not limited to, metal napthenates, metal acetylacetonates (chelates), metal octoates, metal acetates, metal halides, metal imidazole complexes, and metal amine complexes. Other cure accelerators that may be included in the coating formulation include triphenylphosphine, alkyl-substituted imidazoles, imidazolium salts, and onium borates

In some cases, it may be desirable to use more than one type of cure. For example, both cationic and free radical initiation may be desirable, in which case both free radical cure and ionic cure resins can be used in the composition. These compositions would contain effective amounts of initiators for each type of resin. Such a composition would permit, for example, the curing process to be started by cationic initiation using UV irradiation, and in a later processing step, to be completed by free radical initiation upon the application of heat.

If the coating material contains solvent it will typically require a drying and/or B-staging step. As used herein, “B-staging” (and its variants) is used to refer to the processing of a material by heat or irradiation so that if the material is solubilized or dispersed in a solvent, the solvent is evaporated off with or without partial curing of the material, or if the material is neat with no solvent, the material is partially cured to a tacky or more hardened state. For example, if the material is a flow-able adhesive, B-staging will provide extremely low flow without fully curing, such that additional curing may be performed after the adhesive is used to join one article to another. The reduction in flow may be accomplished by evaporation of a solvent, partial advancement or curing of a resin or polymer, or both. The time and temperature required to achieve this will vary according to the solvent and composition used and can be determined by the practitioner without undue experimentation. The drying and/or B-staging may be done as a step separate from the curing of the end use composition, or it may be done as a separate process step.

If the composition does not contain solvent it may still be desirable to B-stage, or partially advance, the material. This may be done prior to cure to effect hardening of the coating to a non-tacky state so that additional processing may be done before the coating is fully cured.

The coating may or may not require curing, depending on the purpose and composition of the coating. If the coating does require curing the cure may be accomplished either as an individual process step, or in conjunction with another processing operation such as solder reflow. The cure may be done at the wafer level or at the die level, depending on the purpose of the coating, the composition of the coating, and the manufacturing process employed.

If a curing step is utilized, the cure temperature will generally be within a range of 80°-250° C., and curing will be effected within a time period ranging from few seconds or up to 120 minutes, depending on the particular resin chemistry and curing agents chosen. The time and temperature curing profile for each composition will vary, and different compositions can be designed to provide the curing profile that will be suited to the particular industrial manufacturing process.

Depending on the end application, one or more fillers may be included in the composition and usually are added for improved rheological properties and stress reduction. Examples of suitable nonconductive fillers include alumina, aluminum hydroxide, silica, vermiculite, mica, wollastonite, calcium carbonate, titania, sand, glass, barium sulfate, zirconium, carbon black, organic fillers, and halogenated ethylene polymers, such as, tetrafluoroethylene, trifluoroethylene, vinylidene fluoride, vinyl fluoride, vinylidene chloride, and vinyl chloride. Exemplary electrically or thermally conductive fillers include carbon black, graphite, gold, silver, copper, platinum, palladium, nickel, aluminum, silicon carbide, boron nitride, diamond, and alumina.

The filler particles may be of any appropriate size ranging from nano size to several mm. The choice of such size for any particular end use is within the expertise of one skilled in the art. When used in a formulation, fillers typically are present in an amount from 0 to 95%, preferably 20 to 85%, by weight of the total composition.

It is desirable for some compositions to add a fluxing agent to remove metal oxides and prevent re-oxidation of electrical solder joints or of metallic substrates. Fluxing agent selection will depend on the resin chemistry and metallurgy presented. Some of the key requirements of the fluxing agent are that it, and fluxing residues generated by the fluxing process, should not affect the curing of the oligomers or resins present in the composition, should not be too corrosive, and should not out-gas to a detrimental level during heating cycles.

Examples of suitable fluxing agents include compounds that contain one or more hydroxyl groups (—OH), or carboxylic (—COOH) groups or both, such as are present in organic carboxylic acids, anhydrides, and alcohols. Exemplary fluxing agents are, for example, rosin gum, dodecanedioic acid (commercially available as Corfree M2 from Aldrich), sebasic acid, polysebasic polyanhydride, maleic acid, hexahydrophthalic anhydride, methyl hexahydrophthalic anhydride, ethylene glycol, glycerin, tartaric acid, adipic acid, citric acid, malic acid, glutaric acid, glycerol, 3-[bis(glycidyl oxy methyl)methoxy]-1,2-propane diol, D-ribose, D-cellobiose, cellulose, 3-cyclo-hexene-I,I-dimethanol; amine fluxing agents, such as, aliphatic amines having 1 to 10 carbon atoms, e.g., trimethylamine, triethylamine, n-propylamine, n-butylamine, isobutylamine, sec-butylamine, t-butylamine, n-amylamine, sec-amylamine, 2-ethylbutylamine, n-heptylamine, 2-ethylhexylamine, n-octylamine, and t-octylamine; epoxy resins employing a cross-linking agent with fluxing properties. Fluxing agents may also be compounds that chelate with a metal substrate. Fluxing agents will be present in an effective amount, and typically an effective amount ranges from 1 to 30% by weight.

In some compositions it may be desirable to add a coupling agent to the composition. Suitable coupling agents are epoxy silanes, amine silanes agent, or mercapto silanes. Coupling agents, if used, will be used in an effective amount, and a typical effective amount is an amount up to 5% by weight.

For some applications, the composition may also contain a surfactant. Suitable surfactants include organic acrylic polymers, silicones, polyethylene glycol, polyoxyethylene/polyoxypropylene block copolymers, ethylene diamine based polyoxyethylene/polyoxypropylene block copolymers, polyol-based polyoxyalkylenes, fatty alcohol-based polyoxyalkylenes, fatty alcohol polyoxyalkylene alkyl ethers, and mixtures thereof. Surfactants, if used, will be used in an effective amount, and a typical effective amount is an amount up to 5% by weight.

Wetting agents also may be included in the composition. Wetting agent selection will depend on the application requirements and the resin chemistry utilized. Wetting agents, if used, will be used in an effective amount and a typical effective amount is up to 5% by weight. Examples of suitable wetting agents include Fluorad FC-4430 Fluorosurfactant available from 3M, Clariant Fluowet OTN, BYK W-990, Surfynol 104 Surfactant, Crompton Silwet L-7280, Triton X100 available from Rhom and Haas, Propylene glycol with a preferable Mw greater than 240, Gama-Butyrolactone, castor oil, glycerin or other fatty acids, and silanes.

A flow control agent also may be included in the composition. Flow control agent selection will depend on the application requirements and resin chemistry employed. Flow control agents, if used, will be present in an effective amount: an effective amount is an amount up to 5% by weight. Examples of suitable flow control agents include Cab-O-Sil TS720 available from Cabot, Aerosil R202 or R972 available from Degussa, fumed silicas, fumed aluminas, or fumed metal oxides.

Some compositions may include an adhesion promoter, and selection of an appropriate adhesion promoter will depend on the application requirements and resin chemistry employed. Adhesion promoters, if used, will be used in an effective amount and an effective amount is an amount up to 5% by weight. Examples of suitable adhesion promoters include: silane coupling agents such as Z6040 epoxy silane or Z6020 amine silane available from Dow Corning; A186 Silane, A187 Silane, A174 Silane, or A1289 available from OSI Silquest; Organosilane SI264 available from Degussa; Johoku Chemical CBT-1 Carbobenzotriazole available from Johoku Chemical; functional benzotriazoles; thiazoles; titanates; and zirconates.

An air release agent (defoamer) is another optional component to the composition. Air release agent selection will depend on the application requirements and resin chemistry employed. Air release agents, if used, will be used in an effective amount and an effective amount will be an amount up to 5% by weight. Examples of suitable air release agents include Antifoam 1400 available from Dow Corning, DuPont Modoflow, and BYK A-510.

In some embodiments these compositions are formulated with tackifying resins in order to improve adhesion and introduce tack; examples of tackifying resins include naturally-occurring resins and modified naturally-occurring resins; polyterpene resins; phenolic modified terpene resins; coumarons-indene resins; aliphatic and aromatic petroleum hydrocarbon resins; phthalate esters; hydrogenated hydrocarbons, hydrogenated rosins and hydrogenated rosin esters.

In some embodiments other components may be included, for example, diluents such as liquid polybutene or polypropylene; petroleum waxes such as paraffin and microcrystalline waxes, polyethylene greases, hydrogenated animal, fish and vegetable fats, mineral oil and synthetic waxes, naphthenic or paraffinic mineral oils.

In other embodiments, monofunctional reactive diluents can be included to incrementally delay an increase in viscosity without adversely affecting the physical properties of the cured coating. Suitable diluents include p-tert-butyl-phenyl glycidyl ether, allyl glycidyl ether, glycerol diblycidyl ether, glycidyl ether of alkyl phenol (commercially available from Cardolite Corporation as Cardolite NC513), and Butanediodiglycidylether (commercially available as BDGE from Aldrich). Preferred diluents are the reactive diluents disclosed earlier in this specification.

Other additives, such as stabilizers, antioxidants, impact modifiers, and colorants, in types and amounts known in the art, may also be added to the formulation.

Common solvents that readily dissolve the resins, and with a proper boiling point ranging from 25° C. to 200° C. can be used for various applications. Examples of solvents that may be utilized include ketones, esters, alcohols, ethers, and other common solvents that are stable. Suitable solvents include γ-butyrolactone, propylene glycol methyl ethyl acetate (PGMEA), and 4-methyl-2-pentanone.

Curing can take place by thermal exposure, ultraviolet (UV) or microwave irradiation, or a combination of these. Curing conditions will be tailored to the specific formulation and can be readily determined by the practitioner. Furthermore, the composition may be B-stageable or not, depending on the application requirements.

### Examples

### Example 1

### Maleimide/Thiol Oligomer

A maleimide/thiol oligomers was prepared by the Michael addition reaction of one part by weight of 4,4′-bismercaptophenyl sulfide (MPS), to eight parts by weight of BMI-1 using 3-aminopropyltrimethoxy silane as catalyst.

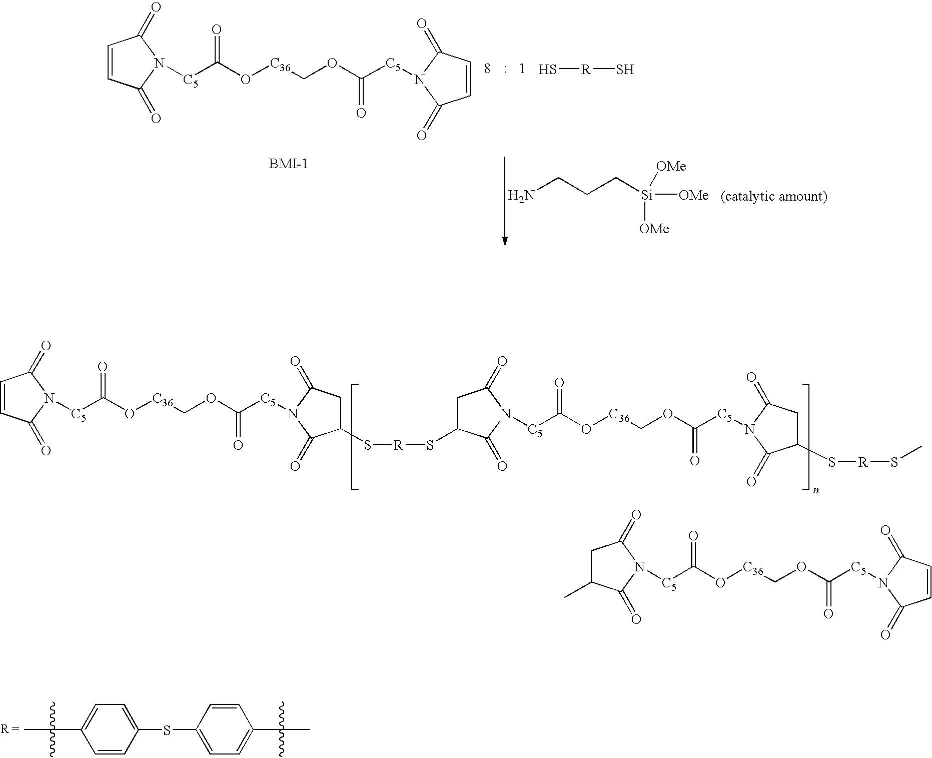

The bismaleimide BMI-1 (55 g, 60.0 mmol) and tetrahydrofuran (66 mL) were charged into a 250 mL reaction flask equipped with an overhead stirrer and condenser. MPS (1.98 g, 7.9 mmol) was added and the mixture stirred until a homogeneous solution was obtained. Then, 4-methoxyphenol (15.3 mg, 0.12 mmol), aminopropyltrimethoxy silane (26 μL), and GENORAD 16 (88 μL, polymerization inhibitor in acrylic acid ester, trademark of and sold by Rahn USA Corporation) were added. The mixture was stirred again until homogeneous. The reaction flask submerged into an oil bath at 85° C. and the reaction solution continuously refluxed for eight hours. The reaction mixture was cooled to ambient temperature and the solvent removed in vacuum. The yield was approximately 97%. The product had a viscosity of 7500 cPs at 25° C. compared to a viscosity of the BMI-1 of 2600 cPs at 25° C. The excess amount of BMI-1 was used to ensure the consumption of the thiol, the presence of which could lead to poor work life when the reaction mix is formulated into an adhesive with a peroxide initiator.

### Example 2

### Maleimide/Thiol Oligomer

A maleimide/thiol oligomer was prepared with the same reactants as in Example 1, except that the ratio of thiol to bismaleimide was 1:4.

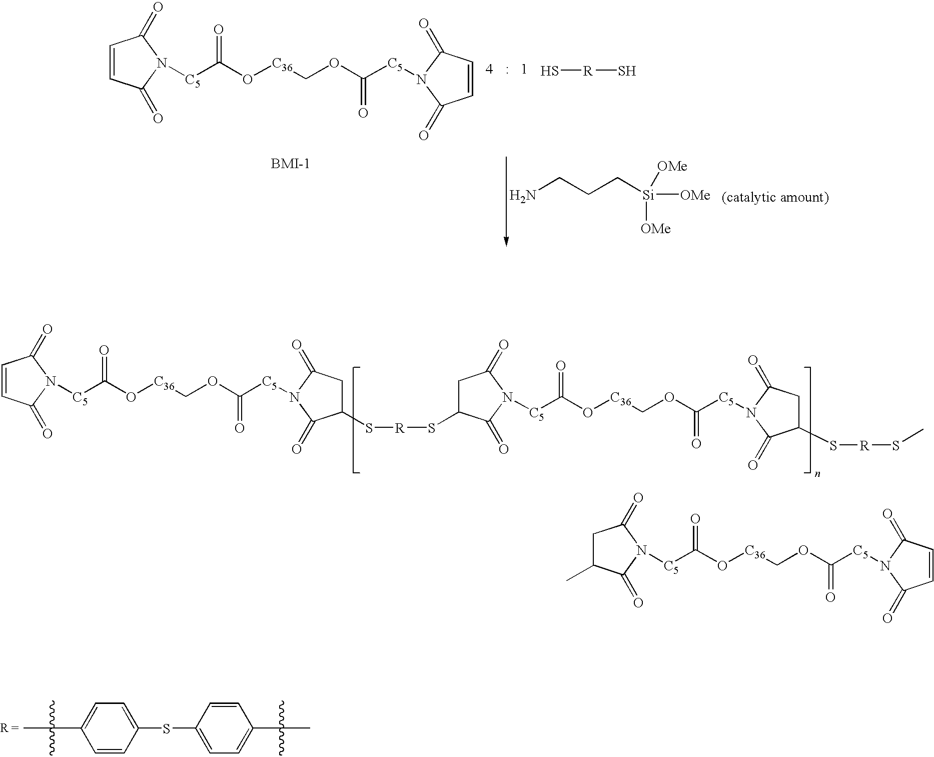

The bismaleimide BMI-1 (60 g, 65.2 mmol) and tetrahydrofuran (70 mL) were charged into a 250 mL reaction vessel equipped with an overhead stirrer and condenser. MPS (4.08 g, 16.3 mmol) was added and the mixture stirred until a homogeneous solution was obtained. Then, 4-methoxyphenol (15.3 mg, 0.12 mmol), aminopropyltrimethoxy silane (45 μL), and GENORAD 16 (150 μL) were added. The mixture was stirred again until homogeneous. The reaction flask was submerged into an oil bath at 85° C. and the reaction solution continuously refluxed for eight hours. The reaction mixture was cooled to ambient temperature and the solvent was removed in vacuum. The yield was approximately 95%. The viscosity of the product was 23960 cPs at 25° C.

### Example 3

### Maleimide/Thiol Oligomer

A maleimide/thiol oligomer was prepared by the Michael addition reaction of one part by weight of 4,4′-bismercaptoethyl sulfide (MES), to eight parts by weight BMI-1.

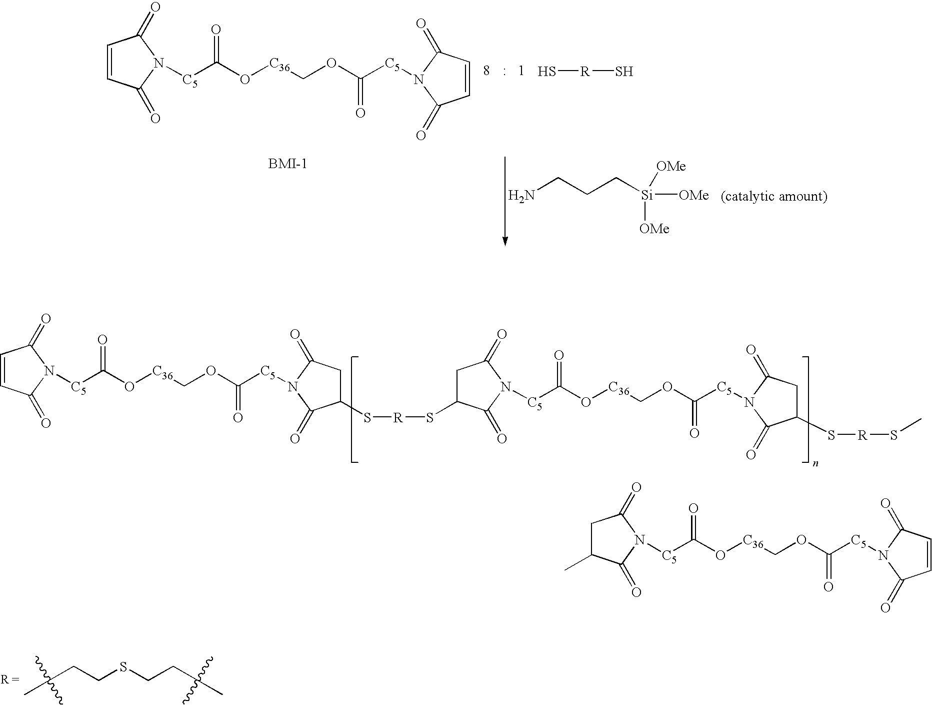

The bismaleimide BMI-1 (24-405A, 100 g, 109 mmol) and tetrahydrofuran (100 mL) were charged into a 500 mL reaction flask equipped with an overhead stirrer and condenser. MES (1.78 mL, 13.6 mmol was added and the mixture stirred until a homogeneous solution was obtained. Then, 4-methoxyphenol (30 mg, 0.24 mmol), aminopropyltrimethoxy silane (52 μL), and GENORAD 16 (160 μL) were added. The mixture was stirred again until homogeneous. The reaction flask was submerged into an oil bath at 85° C. and the reaction solution continuously refluxed for 12 hours. The reaction mixture was cooled to ambient temperature and the solvent was removed in vacuum. The yield was approximately 95%.

### Example 4

### Maleimide/Thiol Oligomer

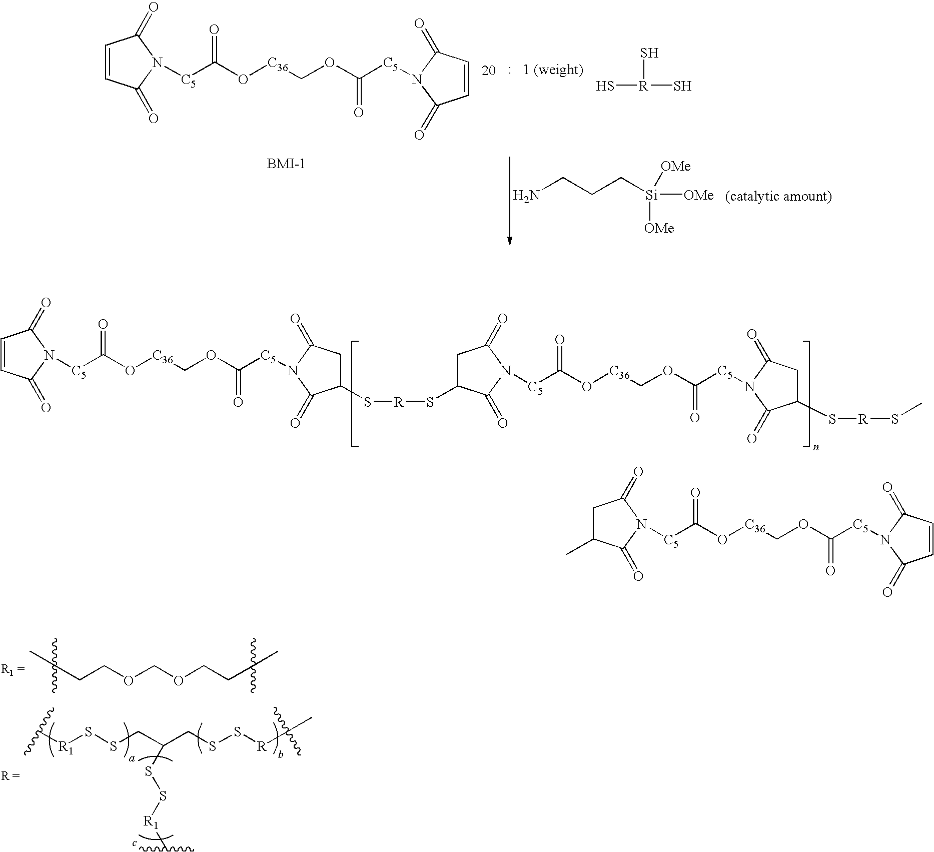

A maleimide/thiol oligomer was prepared by the Michael addition reaction of one part by weight of a thiol sold under the tradename THIOPLAST G4 by Ackros and 20 parts by weight of BMI-1.

Bismaleimide BMI-1 (60 g, 65.2 mmol) and tetrahydrofuran (66 mL) were charged into a 250mL reaction vessel equipped with an overhead stirrer and condenser. Thiol, THIOPLAST G4 (3.0 g), was added and the mixture stirred until a homogeneous solution was obtained. Then, 4-methoxyphenol (16.7 mg, 0.13 mmol), aminopropyltrimethoxy silane (10.0 μL), and GENORAD 16 (30 μL) were added and the mixture stirred until homogeneous. The reaction flask was submerged into an oil bath at 85° C. and the reaction solution continuously refluxed for ten hours. The reaction contents were cooled to ambient temperature and the solvent removed in vacuum. The yield was approximately 95%.

### Example 5

### Maleimide/Thiol Oligomer

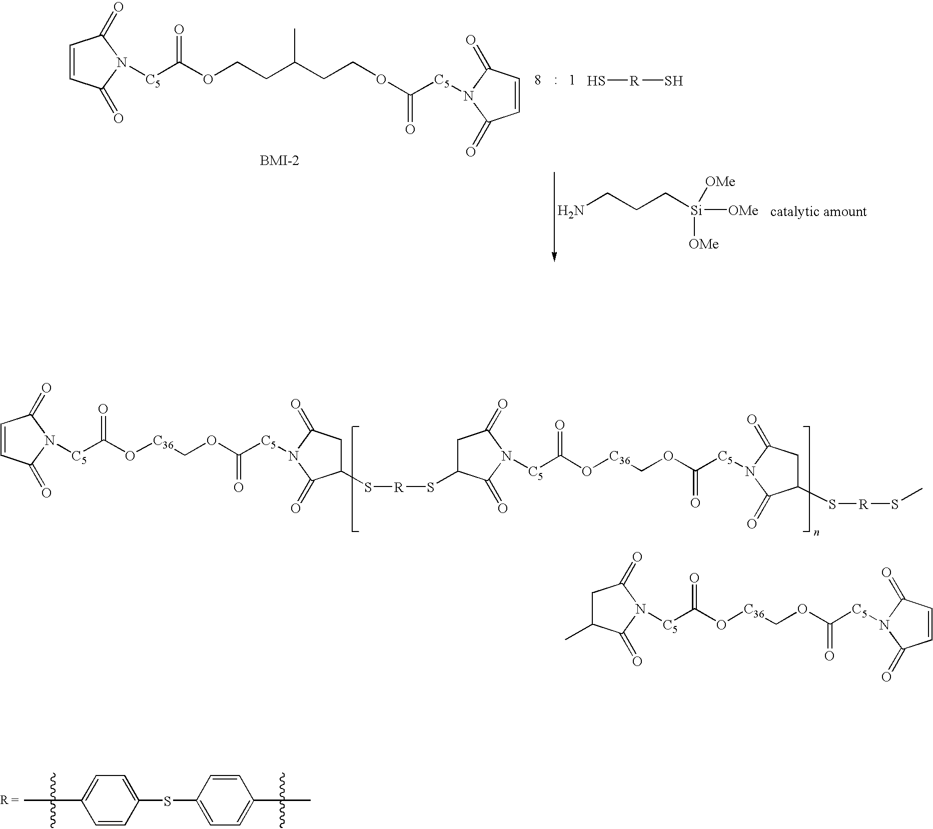

A maleimide/thiol oligomer was prepared by the Michael addition reaction of one part by weight of 4,4′-bismercaptophenyl sulfide (MPS), to eight parts by weight of BMI-2, having the structure depicted in the above reaction scheme.

Bismaleimide BMI-2 (50 g, 99 mmol) and tetrahydrofuran (66 mL) were charged into a 250mL reaction vessel equipped with an overhead stirrer and condenser. Thiol, MPS, (3.09 g, 12.4 mmol), was added and the mixture stirred until a homogeneous solution was obtained. Then, 4-methoxyphenol (23.6 mg, 0.19 mmol), aminopropyltrimethoxy silane (41 μL), and GENORAD 16 (136 μL) were added and the mixture stirred until homogeneous. The reaction flask was submerged into an oil bath at 85° C. and the reaction solution continuously refluxed for eight hours. The reaction contents were cooled to ambient temperature and the solvent removed in vacuum. The yield was approximately 95%.

### Example 6

### Maleimide/Thiol Oligomer

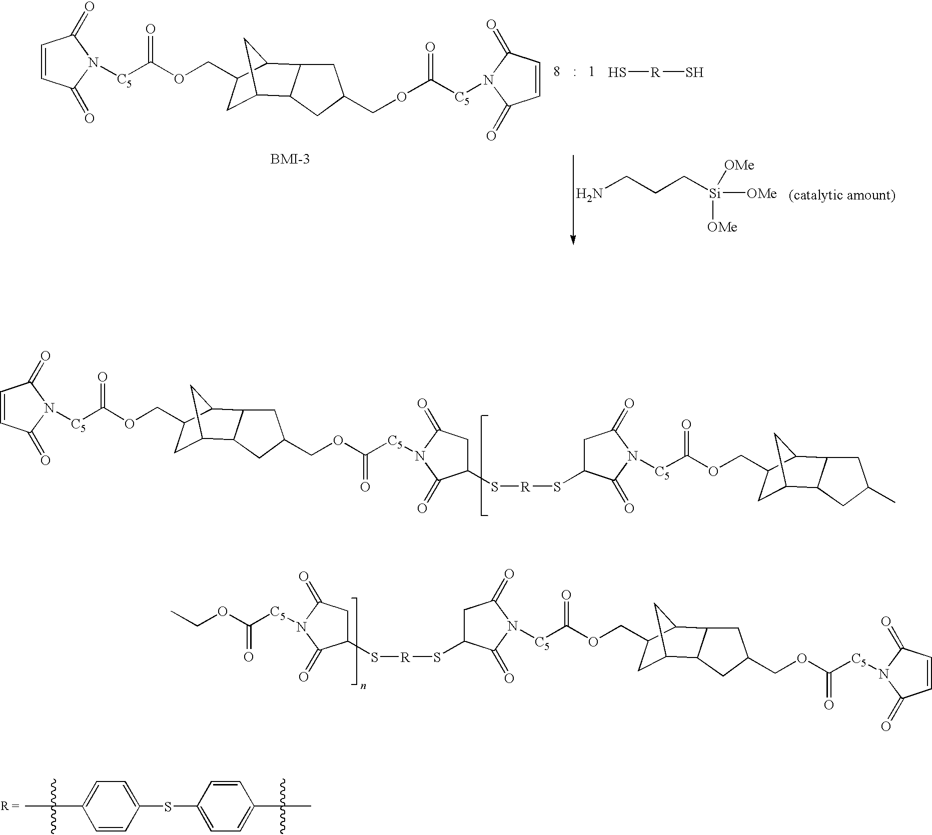

A maleimide/thiol oligomer was prepared by the Michael addition reaction of one part by weight of 4,4′-bismercaptophenyl sulfide (MPS), to eight parts by weight of BMI-3, having the structure depicted in the above reaction scheme.

Bismaleimide BMI-3 (111 g, 200 mmol) and tetrahydrofuran (111 mL) were charged into a 250 mL reaction vessel equipped with an overhead stirrer and condenser. Thiol, MPS (6.3 g, 25.2 mmol), was added and the mixture stirred until a homo-geneous solution was obtained. Then, 4-methoxyphenol (65 mg, 5.0 mmol), aminopropyltrimethoxy silane (50 μL), and GENORAD 16 (150 μL) were added and the mixture stirred until homogeneous. The reaction flask was submerged into an oil bath at 85° C. and the reaction solution continuously refluxed for ten hours. The reaction contents were cooled to ambient temperature and the solvent removed in vacuum. The yield was approximately 95%.

### Example 7

### Adhesive Strength

The inventive Oligomer from Example 1 was formulated into adhesive composition as a partial substitute for BMI-1 and tested for adhesive strength under hot, wet conditions (121° C. saturated steam pressure in a Parr bomb). The adhesion strength was tested on both Ag and PPF (nickel, palladium, gold alloy) metal leadframes with a bare silicon die. The formulations and results of the testing are set out in Table 1. To test for adhesive strength, a bead of each of the formulations was dispensed onto both a silver substrate and a PPF substrate, a 5 mm×5 mm silicon die was placed onto the adhesive bead to produce an approximately 25 micron bondline. Six assemblies for each adhesive formulation and substrate were prepared. Each assembly was cured in an oven at 175° C. for 30 minutes. Each die was sheared from its substrate at 270° C. with a Dage 2400-PC Die Shear Tester. The level of force needed to effect the shear is reported as die shear strength (DSS) in kilogram force. The results were pooled and averaged and are reported in Table 1, together with the formulation for each adhesive. The data show that with increasing level of oligomer in the adhesive composition, there is an improvement in adhesive strength.

TABLE 1

| | 1 | 2 | 3 | 4 |
|---|---|---|---|---|
| BMI Oligomer resin | 5.00 | 4.50 | 4.00 | 3.00 |
| Sulfur containing | | 0.50 | 1.00 | 2.00 |
| oligomer | | | | |
| Mulifunctional | 1.00 | 1.00 | 1.00 | 1.00 |
| crosslinker | | | | |
| Reactive diluent | 7.70 | 7.70 | 7.70 | 7.70 |
| Adhesion promoter 1 | 0.40 | 0.40 | 0.40 | 0.40 |
| Adhesion promoter 2 | 0.50 | 0.50 | 0.50 | 0.50 |
| Cure initiator | 0.40 | 0.40 | 0.40 | 0.40 |
| Silver filler | 85.00 | 85.00 | 85.00 | 85.00 |
| Rheology (cps at 5 RPM) | 8948 | 8948 | 9362 | 9445 |
| Thixotropic IndexI | 4.81 | 5.09 | 4.87 | 4.82 |
| Viscosity % increase | 15.74% | 14.81% | 6.20% | 14.91% |
| after 24 hours | | | | |
| Volume Resistivity | 0.000316 | 4.88E−05 | 5.14E−05 | 5.45E−05 |
| kg DSS at 270° C. on PPF | 18.26875 | 17.54425 | 20.14525 | 29.722 |
| substrate | | | | |
| SD (standard deviation) | 5.115401 | 3.704389 | 13.83143 | 7.967046 |
| kg DSS at 270° C. | 1.848667 | 3.14675 | 8.316 | 10.97167 |
| on PPF substrate | | | | |
| after 16 hours at | | | | |
| 121° C. steam pressure | | | | |
| SD | 0.650946 | 0.844875 | 0.285617 | 2.076213 |
| kg DSS at 270° C. | 7.97475 | 10.19975 | 12.7125 | 18.99425 |
| on Ag substrate | | | | |
| SD | 2.000972 | 4.030677 | 5.045899 | 7.084456 |
| kg DSS at 270° C. | 0.67125 | 3.70375 | 4.9135 | 7.775 |
| on Ag substrate | | | | |
| after 16 hours at | | | | |
| 121° C. steam pressure | | | | |
| SD | 0.405696 | 2.168888 | 2.183959 | 1.226045 |

What is claimed:

1. An oligomeric compound that is the Michael addition reaction product of a thiol with a maleimide compound that has ester functionality.

2. The oligomeric compound according to claim 1 in which the maleimide compound has the structure: 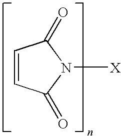
in which n is an integer and X is an aliphatic or aromatic moiety that contains at least one ester functionality.

3. The oligomeric compound according to claim 2 in which the X moieties are selected from the group consisting of: 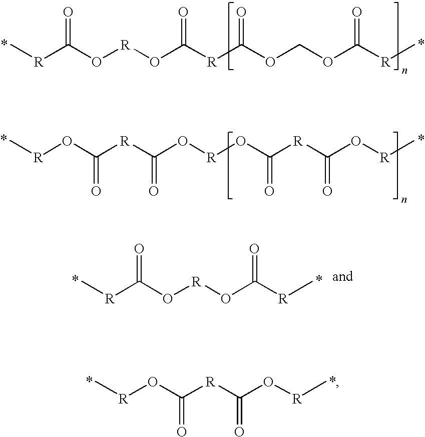
in which each R is independently an aliphatic or aromatic moiety, with or without heteroatoms, or a siloxane having the structure 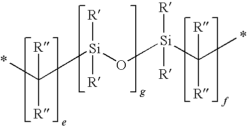
in which R″ is independently for each position a H or an alkyl group having 1 to 5 carbon atoms, and R is independently for each position an alkyl group having 1 to 5 carbon atoms or an aryl group, and e and f independently are an integer from 1 to 10 and g is an integer from 1 to 50.

4. The oligomeric compound according to claim 1 having the structure

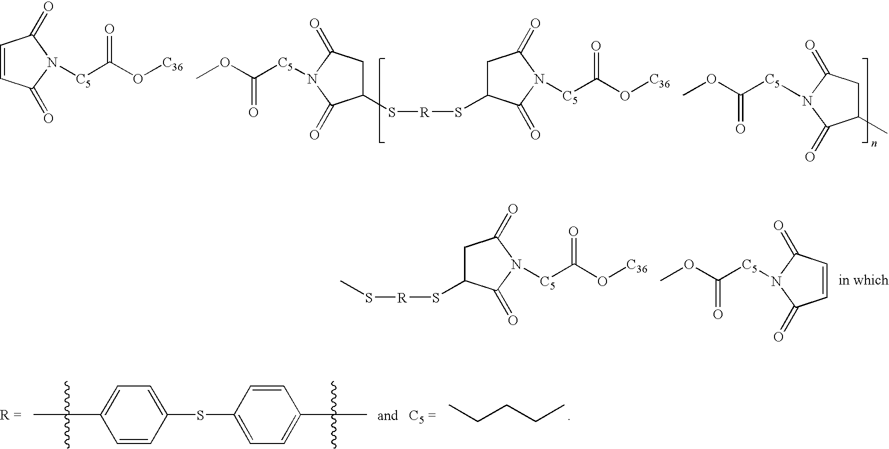

5. The oligomeric compound according to claim 1 having the structure

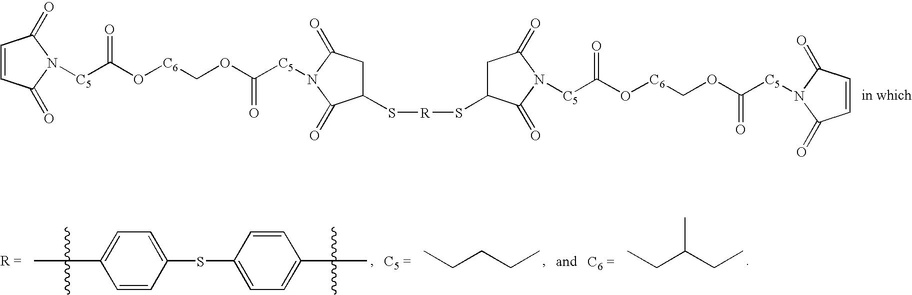

6. The oligomeric compound according to claim 1 having the structure

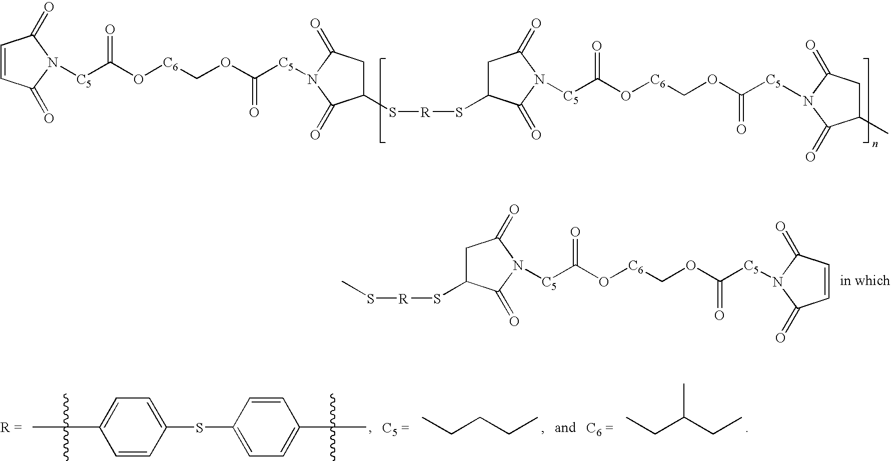

7. The oligomeric compound according to claim 1 having the structure

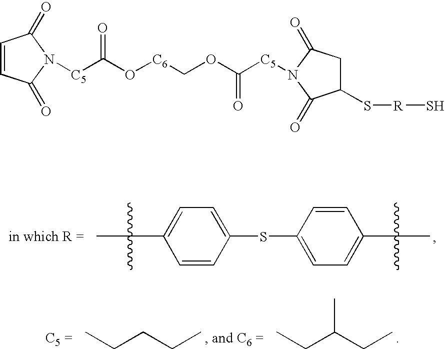

8. The oligomeric compound according to claim 1 having the structure

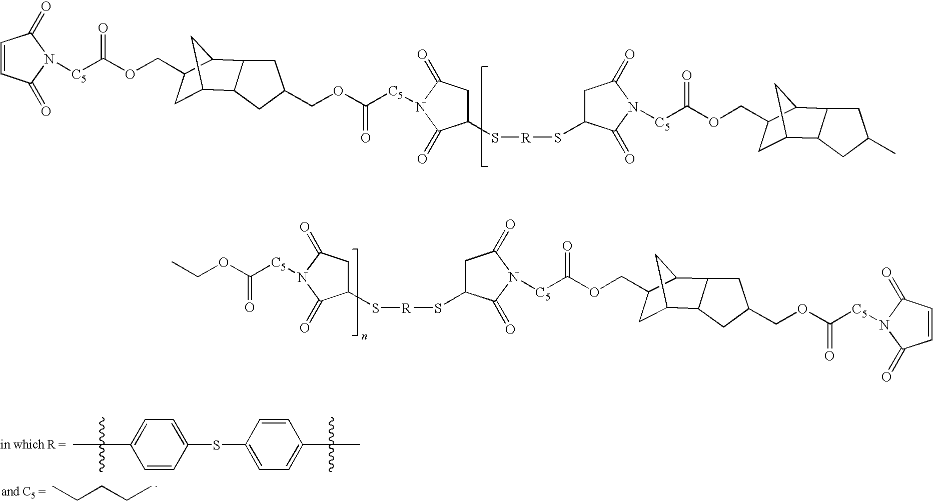

9. The oligomeric compound according to claim 1 having the structure

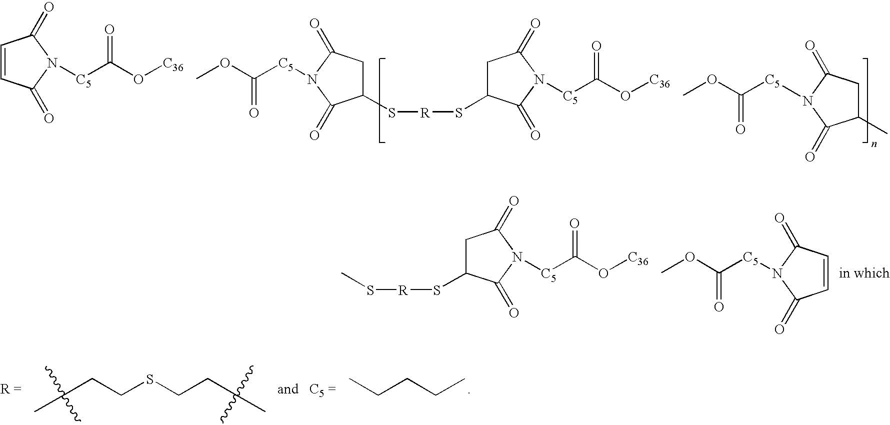

10. The oligomeric compound according to claim 1 having the structure

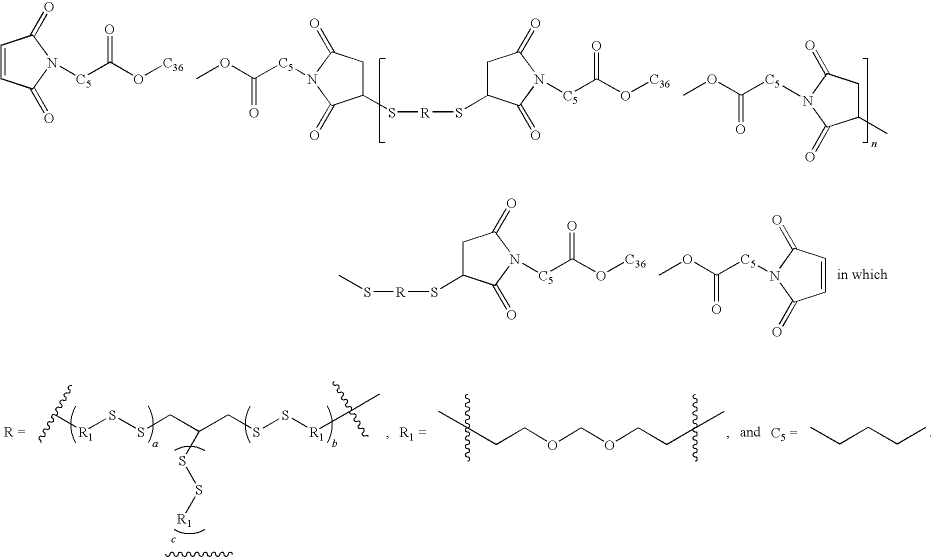

11. The oligomeric compound according to claim 1 having the structure

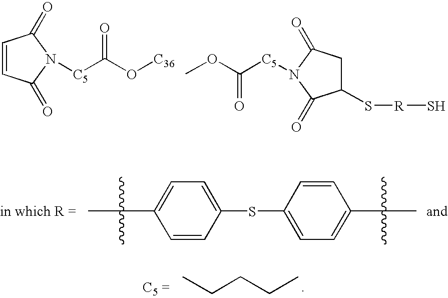

12. The oligomeric compound according to claim 1 having the structure

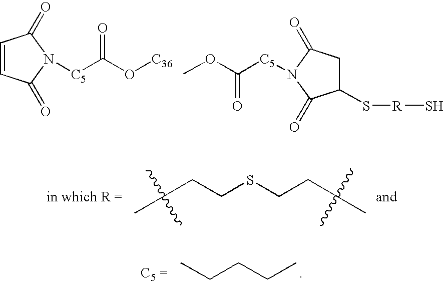

13. The oligomeric compound according to claim 1 having the structure

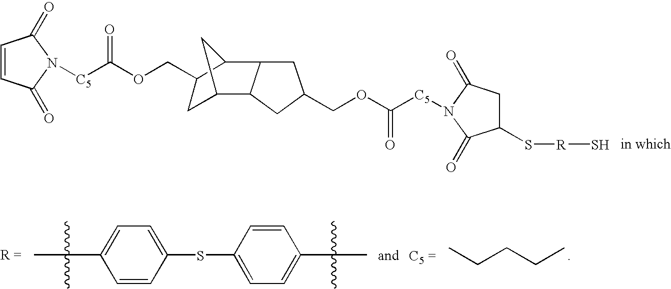

14. The oligomeric compound according to claim 1 having the structure

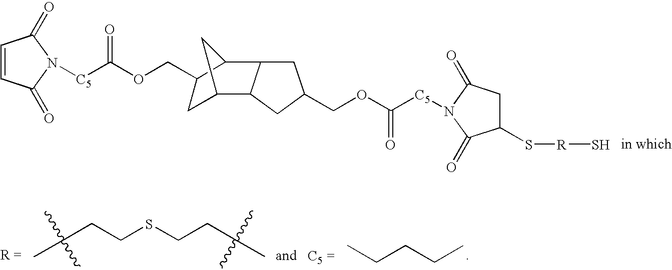

15. The oligomeric compound according to claim 1 having the structure

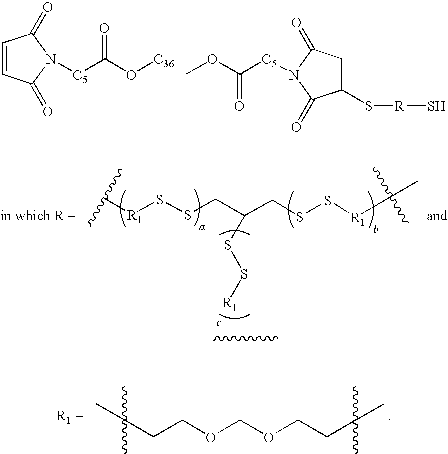

16. The oligomeric compound according to claim 1 having the structure

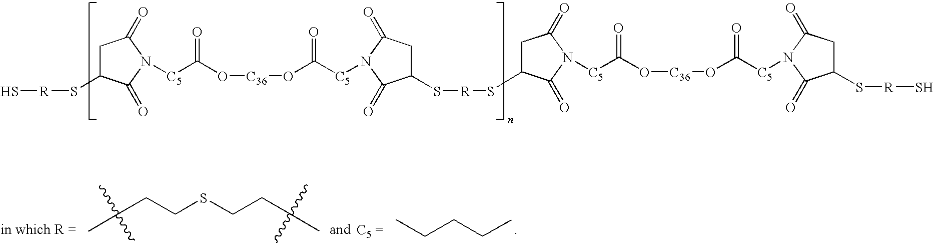

17. The oligomeric compound according to claim 1 having the structure

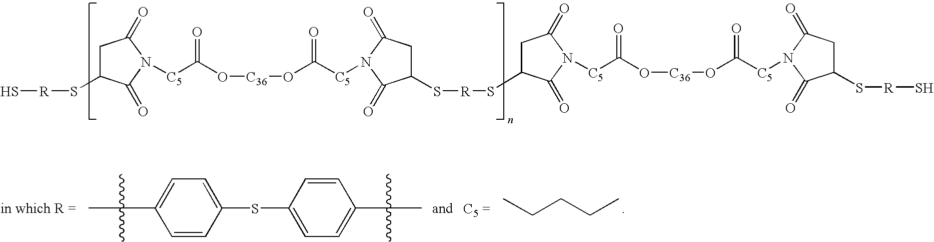

18. A curable composition comprising the oligomeric compound of claim 1.
